(19) United States
(12) Patent Application Publication
(10) Pub. No.: US 2002/0138066 A1
(43) Pub. Date: Sep. 26, 2002

(54) Multiple compartment bag with openable closure assembly

(75) Inventors: Keith Manica, Lakewood, CO (US); Dennis J. Hlavinka, Arvada, CO (US); Jeannie M. Woods, Arvada, CO (US)

Correspondence Address: GAMBRO, INC, PATENT DEPARTMENT, 10810 W COLLINS AVE, LAKEWOOD, CO, 80215, US

(73) Assignee: Gambro, Inc.

(21) Appl. No.: 10/104,766

(22) Filed: Mar. 21, 2002

Related U.S. Application Data
(60) Provisional application No. 60/278,318, filed on Mar. 23, 2001.

(51) Int. Cl. A61B 19/00; A61M 37/00; B65D 35/22

(52) U.S. Cl. 604/410; 604/6.15; 604/522; 222/94

(57) ABSTRACT

A container for pathogen inactivating and storing blood or blood components, which includes multiple sub-compartments fluidly interconnected by an openable closure assembly. Each sub-compartment may contain a component necessary for pathogen inactivation or blood storage. Upon opening the openable closure assembly, the sub-compartments become one compartment and the components contained within each sub-compartment may be mixed together to form a combined solution for pathogen inactivation and/or storage of blood or blood components.

This application claims priority off of U.S. Provisional Patent Application No. 60/278,318, filed Mar. 23, 2001 which is incorporated herein in its entirety to the extent not incompatible herewith.

### FIELD OF THE INVENTION

This invention is generally related to a single bag having multiple sub-compartments for sterilizing and storing solutions used to pathogen inactivate blood or blood components thought to contain pathogens. This invention particularly relates to a bag having an openable closure assembly which when openeded, allows the contents of each sub-compartment to be mixed together. This invention also relates to a bag having features which enable thorough mixing of fluid within the bag. More particularly, this invention relates to a bag having a partial seal or weld, which forms a narrowed portion within the interior of the container to create vortices within the fluid.

### BACKGROUND

Contamination of human blood and blood components with pathogens such as human immunovirus (HIV), hepatitis and/or bacteria create a serious risk for patients who receive blood or blood components via blood transfusions. Whole blood, packed red cells, platelets and plasma (either fresh or fresh frozen) are examples of such transfusable blood and blood component products. To help combat this problem, blood and biologically useful fluids can be decontaminated using pathogen inactivating agents or photosensitizers which, when activated, thereby inactivate pathogens contained in the blood or fluid but do not destroy the biological activity of the blood or fluid components.

The pathogen inactivation agents which may be useful in this invention include the class of photosensitizers known in the art to be useful for inactivating microorganisms. A “photosensitizer” as defined here is any compound which absorbs radiation of one or more defined wavelengths and subsequently transfers the absorbed energy to an energy acceptor. Thus, such photosensitizers may be activated by the application of electromagnetic spectra (e.g., light) to then inactivate certain pathogens with which they may interact. Non-photosensitized pathogen inactivation agents are also considered within the realm of the present invention.

Various photosensitizers have been proposed for use as blood or blood component additives to inactivate pathogens in body fluids. Examples of non-endogenous photosensitizers that have been proposed for use as blood or blood component additives include porphyrins, psoralens, acridines, toluidines, flavins (acriflavin hydrochloride), phenothiazine derivatives, coumarins, quinolines, quinones, anthroquinones and dyes such as neutral red and methylene blue.

Other categories of photosensitizers are endogenous pathogen inactivation agents, such as 7,8,10-trimethylisoalloxazine (lumiflavin), 7,8-dimethylalloxazine (lumichrome), isoalloxazine-adenine dinucleotide (flavin adenine dinucleotide [FAD]), alloxazine mononucleotide (flavin mononucleotide [FMN] and riboflavin-5-phosphate), vitamin K and vitamin L and their metabolites and precursors, napththoquinones, naphthalenes and naphthols as well as their derivatives. One preferred example of an endogenous photosensitizer contemplated for use with this invention is an alloxazine such as 7,8-dimethyl-10-ribityl isoalloxazine, commonly known as riboflavin. An advantage of using endogenous photosensitizers to inactivate blood contaminants is that endogenous photosensitizers are not inherently toxic to the blood cells and if photoactivated do not yield toxic photoproducts after radiation. Therefore, no removal or purification step is required after the decontamination process, and the treated product then can be stored in the same solution used in the pathogen inactivation process, transfused into a patient, or returned directly to a donor's body.

One method of decontaminating blood or blood components includes mixing an effective amount of a photosensitizer with the fluid to be decontaminated in a batch-wise way; then exposing the fluid to an amount of photoradiation at an appropriate wavelength sufficient to activate the photosensitizer and allow the activated agent to interfere with the pathogens contained within the fluid such that the pathogens contained in the fluid are inactivated. In pathogen inactivation examples, the wavelength of light used will depend on the photosensitizing agent selected. The light source or sources may provide light in the visible range, the ultraviolet range, or a mixture of light in both the visible and the ultraviolet ranges.

Decontamination systems may be designed as stand-alone units as described above, or may readily be incorporated into existing apparatuses known to the art for separating or treating blood to be withdrawn from or administered to a patient. For example, such blood-handling apparatuses include the COBE Spectra™ or TRIMA® apheresis systems, available from Gambro BCT Inc., Lakewood, Colo., as well as the apheresis systems of other manufactures. The decontamination system may be inserted before the collected blood is separated into components. The decontamination system may also be inserted downstream of the point where the blood is separated and collected just prior to reinfusion of the blood product back into the patient, or at any point after separation of blood constituents. It is further understood that discrete irradiation sources could be placed downstream from the collection points of each separated blood component, such as red blood cells, platelets, and plasma. The use of three separate blood decontamination systems, one for each separated blood component, is preferred to placement of a single blood decontamination system upstream of the blood separation vessel of an apheresis system because the lower flow rates in the separate component lines allows for greater ease of irradiation. In other embodiments, decontamination systems for use with the present invention may be used to process previously collected and stored blood products, as well as whole blood, in a batch-wise way, as discussed above.

In some photosensitizer methods, the blood component to be decontaminated is flowed through an entry port into a photopermeable bag or like container. The term “photopermeable” means that the material of the container is adequately transparent to photoradiation. Either before or after the addition of the material to be decontaminated, the photopermeable container may contain a prepackaged fluid used in the photoinactivation process and post-photoinactivation storage.

After the pathogen inactivation process, the pathogen inactivated fluid may then flow out of the photoinactivation container into a storage container through an exit port, or may be stored in the same photopermeable container used in the photoinactivation process until transfused into a patient.

Polymeric bags and like containers are useful as photopermeable bags and such bags are typically constructed from one or more flexible sheets of a polymeric material such as PVC or a polyolefin, which may then be welded together.

A prepackaged photoinactivation solution may be contained in a photopermeable or a polymeric bag or container before the addition of or in addition to blood or blood components, and the solution must be both sterile and maintained in a stable condition. It is possible that during the process of fluid sterilization of the container, or during any long term storage of the photoinactivation solution within the container, before the addition of, or addition to the blood or blood components to be photoinactivated, some components of the photoinactivation solution may interact with other components of the solution, causing a chemical interaction and subsequent degradation of the solution. This may be an undesirable condition as it may make the photoinactivation solution contained within the bag not viable for use in the photoinactivation process. In the preferred embodiment, when the additive or storage solution contains a photosensitizer, it is preferred that the photosensitizer mixture be kept separate from a buffer mixture during sterilization to prevent degredation of one or more components. More specifically, the photosensitizer mixture should be sterilized at a lower pH than that of the buffer mixture. In the past, this problem has been avoided by maintaining such solutions in separate bags or containers, as set forth in U.S. Pat. No. 3,874,384, for example. However, this multiple bag method creates a problem of trying to maintain the sterility of all the separated solutions, while increasing the possibility of inadvertently contaminating the solutions due to the many connections required for multiple bag additions. Therefore, having a single container for maintaining separate storage of certain components of the pathogen inactivation solution from other components before combining them in a pathogen inactivation process is a beneficial and important goal.

### BRIEF SUMMARY OF THE INVENTION

The present invention relates to a method and apparatus for storage and preparation of solutions used in inactivating pathogens in a fluid containing pathogens. In one embodiment, the apparatus preferably comprises a single polymeric container having multiple fluid-tight sub-compartments and an openable closure assembly separating the sub-compartments. The openable closure assembly may be an external removable seal assembly such as a channel and removable spline, a tongue and groove seal or a clamp. The openable closure assembly may also be an internal assembly, such as an internal tongue and groove seal assembly. When opened, the openable closure assembly creates a single compartment within the container. The method of using the above-mentioned apparatuses comprises the steps of opening or removing the openable closure assembly to allow all fluids contained within each separate sub-compartment to combine into a single fluid. The single fluid may then be added to the blood or blood component to be pathogen inactivated. Alternatively, the blood or blood components may be added to the combined fluid.

### BRIEF DESCRIPTION OF THE DRAWINGS

FIG. 1 is a schematic view of one embodiment of an apheresis system which can be used in or with the present invention.

FIG. 2 illustrates an extracorporeal tubing circuit, cassette assembly, and collection bag assembly thereof for use with the system of FIG. 1.

FIG. 3 shows a top view of a polymeric container and a removable seal assembly according to one embodiment of the present invention.

FIG. 4 shows an isometric view of the removable seal assembly according to embodiment 3.

FIG. 5 shows a cross-sectional view of the removable seal assembly of FIG. 3 taken along line 501—501 thereof.

FIG. 6 shows a partially broken away isometric view of a polymeric container and the removable seal assembly of FIG. 3, with a portion of the spline lifted for removal.

FIG. 7 shows a top view of a polymeric container after removal of a removable seal assembly.

FIG. 8 shows a cross-sectional view of another embodiment of an alternative removable closure assembly.

FIG. 9 shows a top view of a further alternative embodiment of the herein described invention with an internal tongue and groove seal structure.

FIG. 10 shows a cross-sectional view of a tongue and groove seal structure according to the alternative embodiment of FIG. 9, taken along line 601—601 thereof.

FIG. 11 shows a top view of the fluid vortices created within a bag similar to that shown in FIG. 3 upon application of a pressure causing the fluid to flow in the area of the downward pointing arrow.

FIG. 11A shows a side view of the fluid vortices created within a bag similar to that shown in FIG. 3, where an external clamp (shown in cross-section) is used to create a constriction within the bag.

FIG. 12 shows a top view of another embodiment of a polymeric container and an external removable clamp.

FIG. 13 shows a top view of the polymeric container of FIG. 12 after removal of the clamp.

FIG. 14 shows a top view of an alternative embodiment after removal of a removable closure assembly.

FIG. 15 shows a top view of the alternative embodiment of FIG. 14 with an external removable clamp.

FIG. 16 shows a cross-sectional view of the alternative embodiment of FIG. 15.

FIG. 17 shows a top view of the fluid vortices created within the bag of FIG. 14 upon application of a pressure causing the fluid to flow in the area of the downward pointing arrow.

FIG. 18 shows a top view of the fluid vortices created within the bag of FIG. 14 upon rotation in the direction of the arrows.

FIG. 19 shows a top view of a bag containing fluid and a photosensitizer on a shaker table to agitate the fluid while exposing the fluid to radiation from a light source.

### DESCRIPTION OF THE PREFERRED EMBODIMENTS

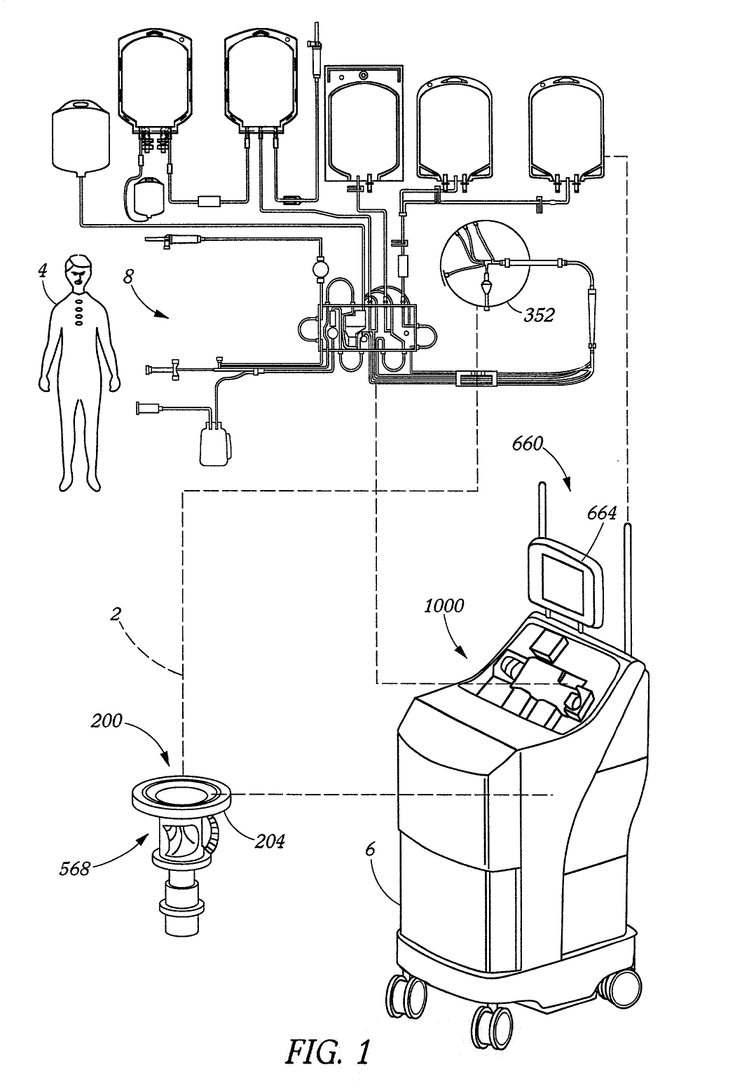

FIG. 1 shows a blood apheresis system 2 for collecting blood components for use with a container of the present invention. Whole blood is withdrawn from a donor/patient 4 and is provided to an apheresis system or blood component separation device 6 where the blood is separated into the various component types and at least one of these blood component types is removed from the device 6. These blood components may then be pathogen inactivated either continuously in a flow-through manner within the apheresis machine 6 or in a separate batchwise step. The pathogen inactivated blood components may then be stored for later use by a patient through transfusion, or may potentially undergo a therapeutic treatment and be returned to the donor/patient 4.

In the blood component separation device 6, blood is withdrawn from the donor/patient 4 and directed through a preconnected disposable set and extracorporeal tubing circuit 8 and a blood-processing vessel 352, which together define a disposable, closed and sterile system. The disposable extracorporeal tubing circuit 8 is mounted on and/or in the blood component separation device 6 which preferably includes a pump/valve/sensor assembly 1000 for interfacing with the extracorporeal tubing circuit 8, and a channel assembly 200 for interfacing with the disposable blood processing vessel 352.

The channel assembly 200 may include a channel housing 204 which is rotatably interconnected with a rotatable centrifuge rotor assembly 568 which provides the centrifugal forces required to separate blood into its various component types by centrifugation. The blood processing vessel 352 may then be interfitted within the channel housing 204. When thus connected as described, blood can flow continuously from the donor 4, through the extracorporeal tubing circuit 8, and into the rotating blood processing vessel 352. The blood within the blood processing vessel 352 may then be continuously separated into various blood component types and at least one of these blood component types (platelets, plasma, white blood cells or red blood cells) is preferably continually removed from the blood processing vessel 352. Blood components which are not being retained for collection or for therapeutic treatment are preferably also removed from the blood processing vessel 352 and returned to the donor 4 via the extracorporeal tubing circuit 8.

Operation of the blood component separation device 6 is preferably controlled by one or more computer processors included therein. In order to assist the operator of the apheresis system with various aspects of its operation, the blood component separation device 6 preferably includes a graphical interface 660 preferably with an interactive touch screen 664.

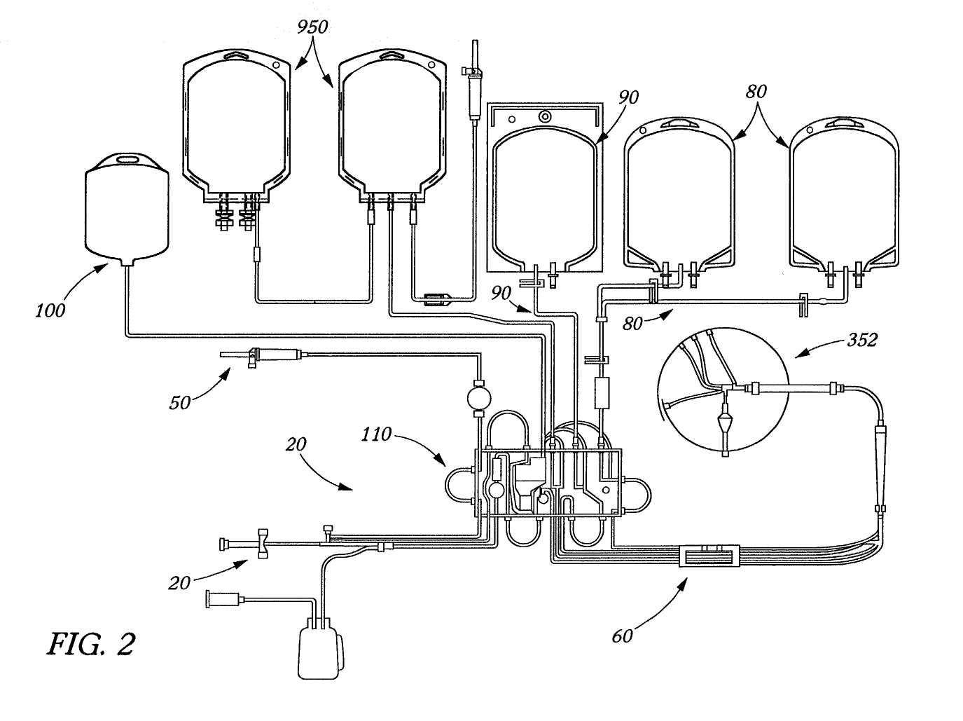

As illustrated in FIG. 2, a preferred preconnected extracorporeal tubing circuit 8 may include a cassette assembly 110 and a number of tubing/collection assemblies 20, 50, 60, 80, 90, 950 and 100 interconnected therewith. Preferably, a blood removal/return tubing assembly 20 provides a single needle interface between a donor 4 (not shown in FIG. 2) and the remainder of the tubing circuit 8 (although a two-needle set-up may also be used). The preferred embodiment includes a cassette assembly 110, which is interconnected between the tubing assembly 20 which connects the donor 4 (not shown in FIG. 2), and blood inlet/blood component tubing line sub-assembly 60 which provides the interface between cassette assembly 110 and blood processing vessel 352. An anticoagulant tubing assembly 50, a platelet collection tubing assembly 80, a plasma collection tubing assembly 90, a red blood cell collection assembly 950 and a vent bag tubing line sub-assembly 100 are also preferably interconnected with cassette assembly 110 in this embodiment. As will be appreciated, the extracorporeal tubing circuit 8 and blood processing vessel 352 are preferably pre-interconnected to combinatively yield a closed, pre-sterilized disposable assembly for a single use.

Most portions of the tubing assemblies 20, 50, 60, 80, 90, 100 and 950 and cassette assembly 110 are preferably made from plastic components including, for example, polyvinyl chloride (PVC) tubing lines, that permit visual observation and monitoring of blood/blood components therewithin during use. It should be noted that thin-walled PVC tubing may be employed for approved, sterile docking (i.e., the direct connection of two pieces of tubing line). In keeping with one preferred aspect of the invention, all tubing lines are preconnected before sterilization of the total disposable assembly to assure that maximum sterility of the system is maintained. It should be noted that a highly desirable advantage to preconnection of all of the elements of the tubing circuit involves the complete pre-assembly and subsequent sterilization after assembly such that no sterile docking is later necessary (spike addition of later added fluids possibly excepted). Thus, the costs and risks of sterile docking may be eliminated.

As mentioned, a cassette assembly 110 in the preferred embodiment, may be mounted upon and operatively interface with the pump/valve/sensor assembly 1000 of blood component separation device 6 during use. Further examples and details of an apheresis system set-up including the loading and interaction of a disposable assembly 8 with a blood component separation device 6, may be found in U.S. Pat. No. 5,653,887; U.S. Pat. No. 5,676,644; U.S. Pat. No. 5,702,357; U.S. Pat. No. 5,720,716; U.S. Pat. No. 5,722,946; U.S. Pat. No. 5,738,644; U.S. Pat. No. 5,750,025; U.S. Pat. No. 5,795,317; U.S. Pat. No. 5,837,150; U.S. Pat. No. 5,919,154; U.S. Pat. No. 5,921,950; U.S. Pat. No. 5,941,842; and No. 6,129,656; among numerous others. A plurality of other known apheresis systems may also be useful herewith, as for example, the Baxter CS-3000 and/or Amicus and/or Autopheresis-C systems, and/or the Haemonetics MCS+ and/or the Fresenius COM.TEC or AS-104 and/or the Dideco or like systems.

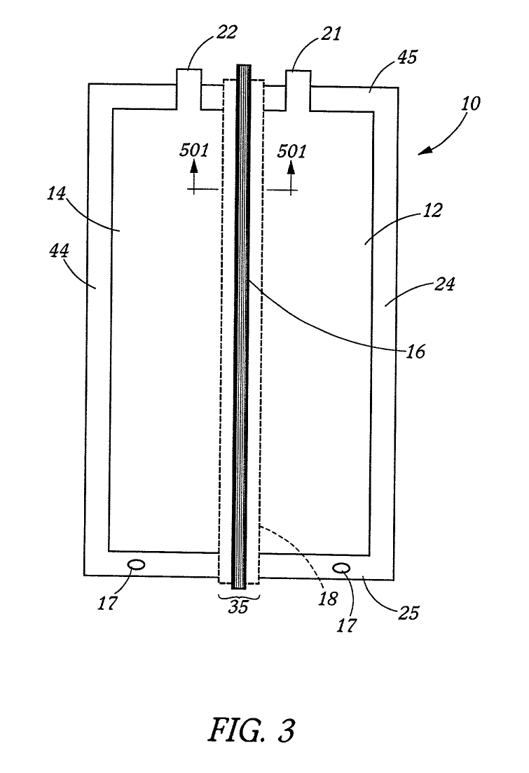

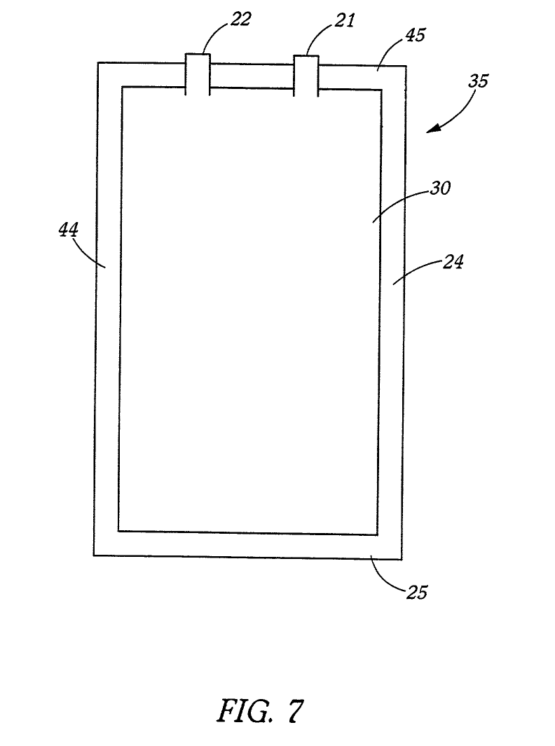

FIG. 3 shows a top plan view of a polymeric container or bag 10 which may be used in accordance with the present invention. It should be noted that like reference numerals in different figures denote like parts. The container 10 is preferentially made of a polymeric type film which is folded over and sealed or welded around its outer border zones during manufacture to form pre-formed seals or welds 24, 25, 44 and 45. The seals or welds 24, 25, 44 and 45 create a fluid tight, sealed interior space or main body compartment (not directly shown in FIG. 3, but see FIG. 7 described below). FIG. 3 shows welds around the entire circumference of the bag. If, as is known in the art, the bag is initially extruded in a substantially tube-like fashion as opposed to a sheet-like fashion, only the top and bottom of the bag need to be sealed or welded to create an entirely closed container. A single polymeric sheet may also be folded over and sealed on three sides to form an entirely closed container as well, without departing from the spirit and scope of the invention. Two ports or openings 21 and 22 allow fluid ingress and egress into and out of the container 10. As shown in FIG. 3, both ports 21 and 22 are preferably located on the same side of the bag 10. However, the ports 21 and 22 may also be located on opposite sides of bag 10 as well, without departing from the spirit and scope of the invention. Furthermore, the bag 10 could contain one or more ports without departing from the spirit and scope of the invention. Any well-known type of port may be used in this invention, including a port having a frangible-type mechanism. The main body compartment (not directly shown) of container 10 is capable of being separated into two distinct fluidly separated sub-compartments 12 and 14 respectively by an openable closure assembly 35. In this embodiment, the openable closure assembly is a removable seal assembly. The removable seal assembly 35 preferably extends along the length of the container 10, also extending across the upper and lower factory seals or welds 25 and 45 of the outer border zones of the bag 10. The length of the removable seal assembly 35 may vary depending on the length of the polymeric bag being used. In FIG. 3 the removable seal assembly 35 is shown disposed along the center of the bag 10 dividing the bag into two substantially equal sub-compartments 12 and 14. However, the removable seal assembly 35 may be used to create unequally sized sub-compartments as well, without departing from the spirit and scope of the present invention. In one embodiment, the removable seal assembly 35 comprises a compressible spline 16 and a substantially rigid trapezoidal channel 18.

The bag 10 may have a hole or holes 17 punched in for example, the lower edge of the pre-formed factory seal 25 to mount the bag 10 in a hanging position. The bag may also not have holes punched in the pre-formed factory seal such as the bag shown in FIG. 7. The bag 10 may be hung before, during or after the process of combining the blood component with the solutions in bag 10. The pre-formed factory seal 25 or any of the other preformed seals may also be made wide enough so that a label describing the contents of each sub-compartment of the bag 10 may be placed on the area over the seal (not shown).

In other embodiments, and as further described below, the external removable seal assembly 35 may also be a clamp, a clip, a tongue and groove seal, or anything of the like which removably divides a single bag into multiple sub-compartments and prevents communication between, or mixing of, the contents contained within each separate sub-compartment until the removable seal assembly is removed. More than one external removable seal assembly may be used to create more than two sub-compartments in a single bag as well.

Bag 10 may be pre-connected and/or post connectable with the extracorporeal tubing circuit 8 as described relative to FIG. 2 above, or may be used separately, as a stand alone apparatus, neither alternative departing from the spirit and scope of the invention.

The bag 10 may also have a connection to a sample bulb via a port (not separately shown) to allow for fluid removal and sample testing or the like. A port which allows for the connection of a spike receptor (not shown) or to enable the sterile docking of a further bag or tube for the addition of a further fluid or blood component may also be added to bag 10. A spike connector would also preferably include a sterile barrier filter as is known in the art.

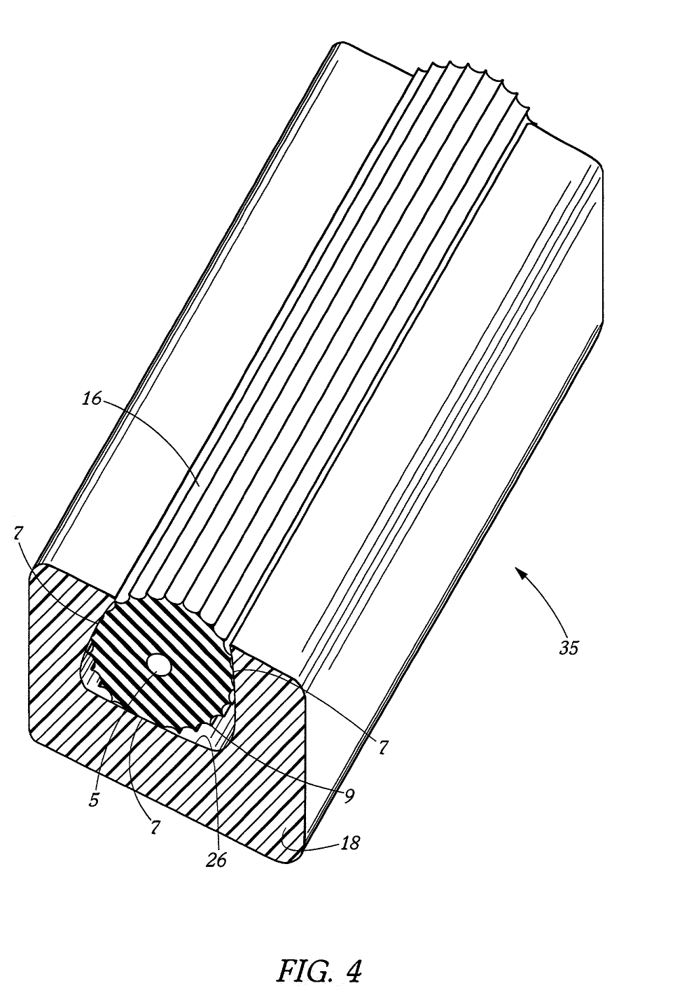

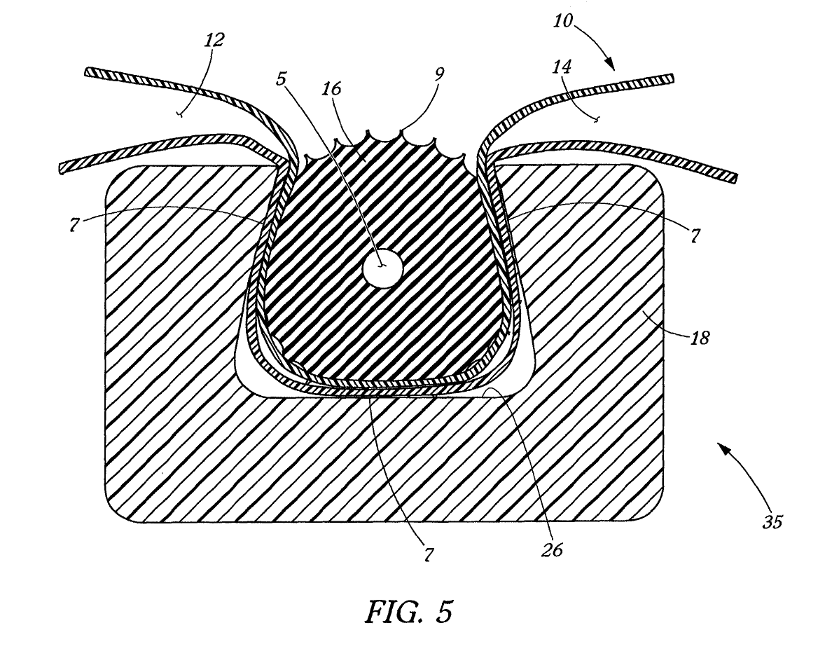

FIG. 4 shows an isometric view of the removable seal assembly 35 (not shown with container 10) according to the present invention. The removable seal assembly 35 preferably includes a removable compressible spline 16 and a channel 18 having an interior trapezoidal shape 26. The compressible spline 16 preferably has surface ridges or ribs 9 along the outer surface as well as a hole 5 running longitudinally through the center of the spline 16. The compressibility of the spline 16 and the surface ridges 9 assist in providing the advantage of creating a fluid tight seal between the spline 16 and the bag 10 (not shown, but see FIG. 5, described below) contained within the channel 18 preferably at three contact points 7. The hole 5 preferably furthers the compressibility of the spline 16 within the channel 18 and thus further helps to create a fluid tight seal between the sub-compartments of the container 10. The removable seal assembly 35 is constructed of materials that will retain both shape and function at temperatures greater than or equal to 250° F., when steam sterilized with the polymeric bag 10 (not shown). Several such channel materials might include polycarbonate and nylon. Several such spline materials might include silicone and polyurethane. However, such examples of suitable materials are not meant to be limiting. Any such material that would comply with the spirit and scope of the invention may be used.

The spline 16 may be constructed without surface ridges 9, and may not have a hole 5 running longitudinally through the center of the spline 16. The spline 16 may also be constructed in a non-circular shape, for example, the spline 16 may have a trapezoidal shape to allow for a more snug fit within the trapezoidal channel 18. The spline may also be constructed of a relatively rigid material.

The trapezoidal channel 18 may also have several alternatives without departing from the original scope of the invention. For example, the interior 26 of the channel 18 may have a non-trapezoidal shape (not shown). The channel 18 may also be flexible, or may be constructed of a semi-rigid material.

FIG. 5 shows a partially broken away cross-sectional view of a removable seal assembly 35 creating separately sealed sub-compartments 12 and 14 in a polymeric container 10. The removable seal assembly 35 creates a temporary, removable seal along the length of the bag, creating two distinct fluidly separate sub-compartments 12 and 14. The removable seal assembly 35 may be easily removed from the bag 10 (as described below) to create a single bag with only one internal compartment (see element 30 of FIG. 7). This is done by simply removing the spline 16 from the trapezoidal channel 18.

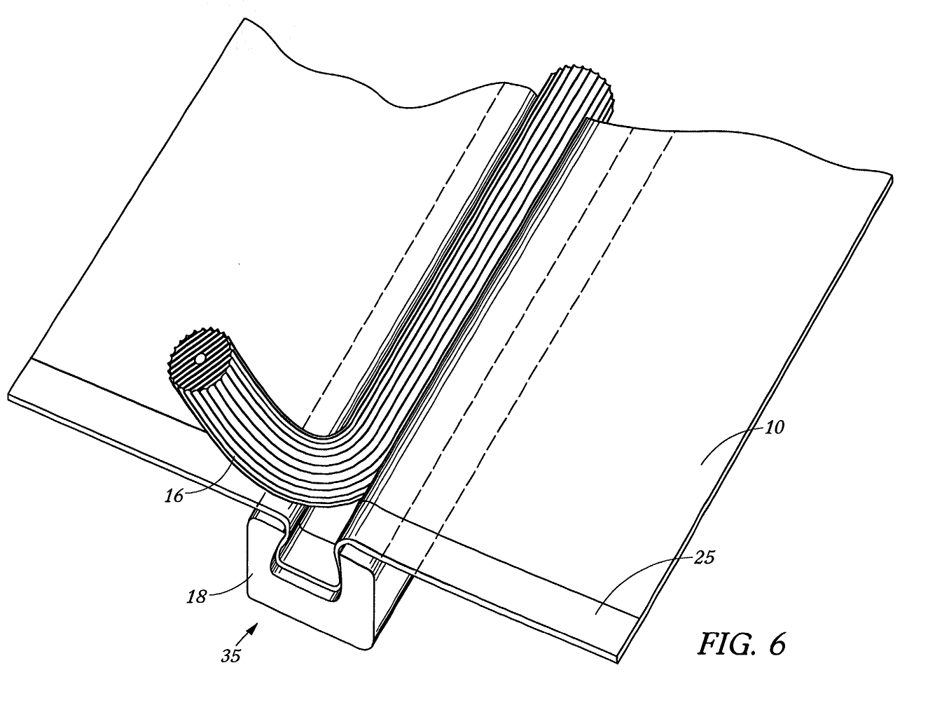

FIG. 6 shows an isometric view of the spline 16 being removed from within the trapezoidal channel 18. The spline 16 may be removed by sliding the spline 16 out of the channel 18, or as shown, by peeling the spline 16 out of the channel at an angle. Once the spline 16 is removed, the bag 10 may be removed from within the trapezoidal channel 18 by peeling the bag 10 out of the channel 18 or by pulling the trapezoidal channel 18 away from the bag 10. Upon the removal of the removable seal assembly 35, the multiple sub-compartments within the bag 10 (see sub-compartments 12 and 14 of FIG. 3) are eliminated and the fluid contained within each sub-compartment 12 and 14 may be mixed together in the single reconstituted compartment (see element 30 of FIG. 7).

FIG. 7 shows a top view of the polymeric container 10 after removal of the removable seal assembly 35. Once the removable seal assembly 35 is removed from the bag 10, the multiple sub-compartments 12 and 14 initially created by the removable seal assembly 35 within the bag 10 are eliminated. The multiple sub-compartments 12 and 14 combine to form a single compartment 30 within the bag 10.

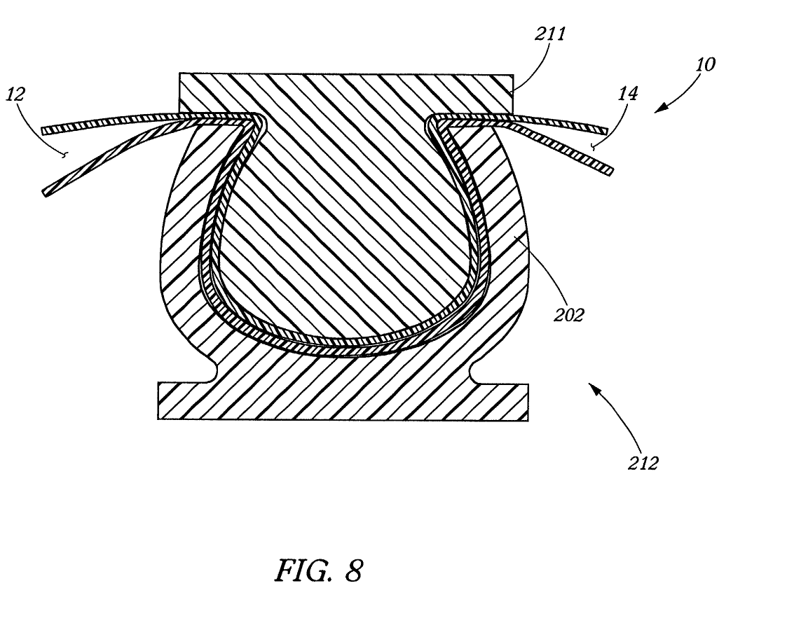

FIG. 8 shows a cross-section of an alternative embodiment of another type of openable closure assembly which enables the creation of separately sealed sub-compartments in a polymeric container 10. The alternative openable closure assembly 212 is an external tongue and groove removable seal structure or assembly. The tongue portion 211 fits into the groove portion 202 to create multiple fluid tight sub-compartments 12 and 14 respectively, within a single bag 10. The tongue portion 211 is made of a substantially rigid material, while the groove portion 202 is made of a substantially flexible material. The tongue portion 211 is placed on the top side of the bag in a location directly opposite the groove portion 202 which is located on the bottom side of the bag. The bag 10 is contained within the groove portion 202, which flexes to accommodate both the bag 10 and the rigid tongue portion 211, creating separately fluidly sealed sub-compartments 12 and 14. Although not shown in FIG. 8, either the tongue portion or the groove portion or both may have ridges or ribs on their respective surfaces to assist in creating a fluid tight seal between the closure assembly and the bag.

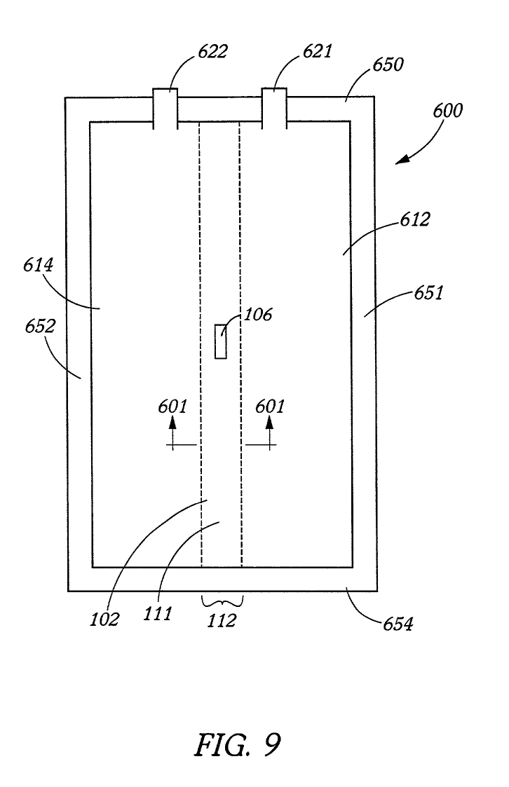

FIG. 9 shows another embodiment of a polymeric container according to the present invention wherein the openable closure assembly is an internal tongue and groove seal assembly. The container 600 is preferentially made of a polymeric type film which is folded over and sealed or welded around its outer border zones during manufacture to form pre-formed seals or welds 650, 651, 652 and 654. The seals or welds 650, 651, 652 and 654 create a fluid tight, sealed interior space or main body compartment (not directly shown). Two ports or openings 621 and 622 allow fluid ingress and egress into and out of the container 600. Although both ports 621 and 622 are shown together on the same side of the container 600, a port could be located on opposite sides of the bag 600 from one another, or may be located on any one of the four sides of the bag. Furthermore, the bag 600 could contain one or more ports without departing from the spirit and scope of the invention. Any well-known type of port may be used in this invention, including a port having a frangible-type mechanism. The main body compartment (not shown) of container 600 is capable of being separated into at least two distinct sub-compartments 612 and 614 respectively, by an internal tongue and groove seal 112. The internal tongue and groove seal structure 112 is shown longitudinally extending through the center of the bag, dividing the bag into two substantially equal sub-compartments 612 and 614. However, the tongue and groove seal structure 112 may be used to create unequally sized sub-compartments as well, without departing from the spirit and scope of the present invention.

The tongue and groove seal structure 112 extends along the length of the container 600, up to the preformed upper or lower seals or welds of the outer border zones 650 and 654 respectively. The length of the tongue and groove seal structure 112 may vary depending on the length of the polymeric bag being used. Furthermore, multiple tongue and groove seal structures may be used to create multiple (more than two) sub-compartments within the single bag. Tabs 106 are preferably located on the external surface of the bag 600 over and/or adjacent to the tongue and groove seal 112. The tabs 106 are shown attached to the bag in the center, but may alternatively be placed anywhere along or adjacent to the tongue and groove seal, without departing from the spirit and scope of the invention.

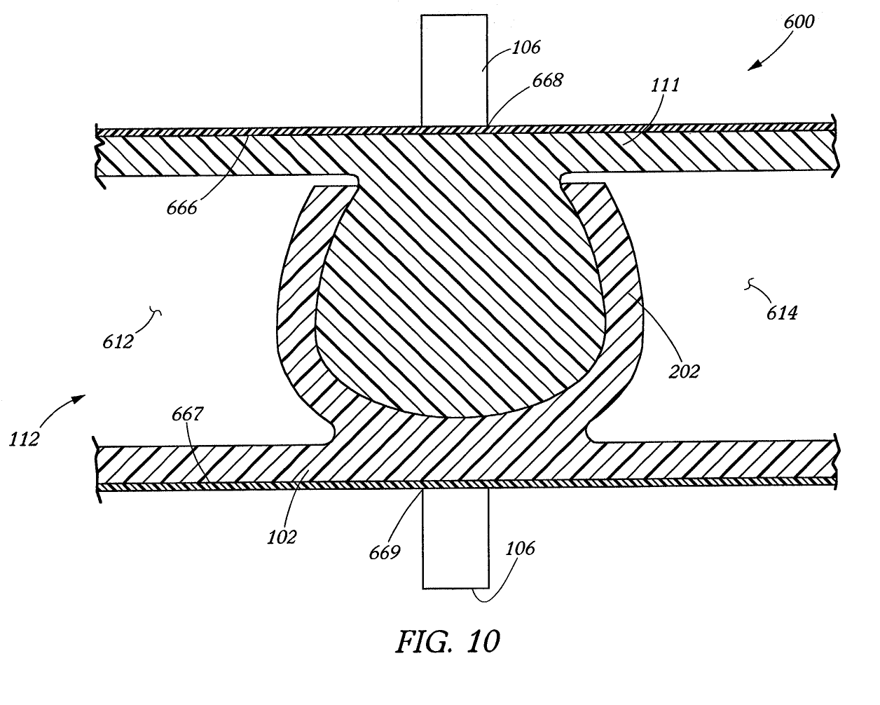

FIG. 10 shows a partially broken away cross-sectional view of an internal tongue and groove seal creating separately sealed sub-compartments within a polymeric container 600, such as that shown in FIG. 9. The internal tongue and groove seal structure 112 creates a temporary, closed seal along the length of the bag, creating two distinct sub-compartments 612 and 614. The tongue portion 111 is affixed to an interior surface 666 of one side of the container 600 while the groove portion 102 is affixed to the opposite interior surface 667 of the container 600. The tongue portion 111 and the groove portion 102 are situated inside the bag directly opposite each other for selective interlocking of the tongue portion 111 into the groove portion 102. Tabs 106 provide for manual grasping of each structural base of the tongue portion and the groove portion to enable the separation of the tongue portion 111 from the groove portion 102. A first tab 106 is connected to the exterior surface 668 of the bag 600 located over or adjacent to the tongue portion 111, and a second tab 106 is connected to the exterior surface 669 of the opposite side of the bag located over or adjacent to the groove portion 102. The tongue portion 111 of the seal assembly 112, and the groove portion 102 of the seal assembly 112, may be easily separated from each other by grasping the tabs 106 and pulling the tabs away from each other, pulling the tongue portion 111 out of the groove portion 102. Although the tabs 106 are shown having a rectangular shape, the tabs may be any shape and size and still comply with the spirit and scope of the invention. For example, the tabs may also be made of string-like material. Any number of tab sets may also be used.

One method of using the above described embodiments is as follows, described in relation to the embodiment of FIG. 3. Although not specifically described, the method may also be used with the alternative embodiments described in FIGS. 8 and 9. Initially, a first solution containing components necessary for pathogen inactivation and/or storage of blood or blood products is prepackaged in sub-compartment 14 of bag 10. A second solution containing other necessary components for pathogen inactivation or other purposes such as storage of blood or separated blood components is prepackaged in sub-compartment 12 of bag 10. The components may be in a dry solid or preferably a liquid form. The components necessary for the solutions may be added to sub-compartment 14 via port 22, and/or to sub-compartment 12 via port 21. The first and second solutions are maintained separately from each other within sub-compartment 14 and sub-compartment 12 via the removable seal assembly 35. In the preferred embodiment, the solution contained within compartment 14 is preferably Solution A as further described below, and the solution contained within compartment 12 is preferably Solution B as also described below.

Solution A may preferably contain an effective amount of a photosensitizer and possibly may also contain an additive. In a preferred embodiment, Solution A contains 7,8-dimethyl-10-ribityl isoalloxazine and glucose. Solution B preferably contains a component such as a buffer and possibly may also contain an anticoagulant. In the preferred embodiment, Solution B contains sodium bicarbonate or sodium phosphate and may also contain sodium citrate. Such packaging allows solutions having different pH's and osmolarities to be stored in the same container. For example, in the preferred embodiment, Solution A has a pH of close to or equal to 3 to 6, whereas Solution B preferably has a pH of between about 7-8. Such packaging also helps to prevent degradation of components such as glucose by allowing such components to be separately sterilized using an apparatus such as that described in patent Application WO 00/24433, filed Oct. 20, 1999. Other well-known heat sterilization methods could also be used. It is also understood that the solutions contained within each sub-compartment do not have to contain all of the above-listed components for pathogen inactivation and/or storage of blood or blood components. Sub-compartments 12 and 14 respectively may contain solutions used solely for blood storage and/or blood collection for example, as well as other blood additive or storage components.

In a preferred use of the invention, after the sterilization of the separately stored solutions has occurred, and shortly before the preliminary mixing of the solutions used in the photoinactivation process is to occur, the removable seal assembly 35 is removed from the container 10, by removing the compressible spline 16 and the trapezoid channel 18 from the bag 10. Removal of the removable seal assembly 35 eliminates the seal or barrier separating the fluids within sub-compartments 12 and 14. With the removal of the removable seal assembly 35, Solution A contained within sub-compartment 14 and Solution B contained within sub-compartment 12 are able to be thoroughly mixed together, forming a single solution possibly containing photosensitizer nutrient, buffer and anticoagulant. After combination of the fluids, the blood or blood component to be pathogen inactivated may be added to container 10 via ports 22 or 23. Alternatively, the combined fluid may be added to a second bag (not shown) containing the blood or blood component to be pathogen inactivated. The blood or blood component to be pathogen inactivated and the pathogen inactivation solution are mixed together, and the mixture may be exposed to a light source. As can be appreciated, a dry solid may be located in sub-component 12 or 14 and can be mixed with either another dry solid or a fluid to make a mixture which may be added to blood or blood components in accordance with the method above.

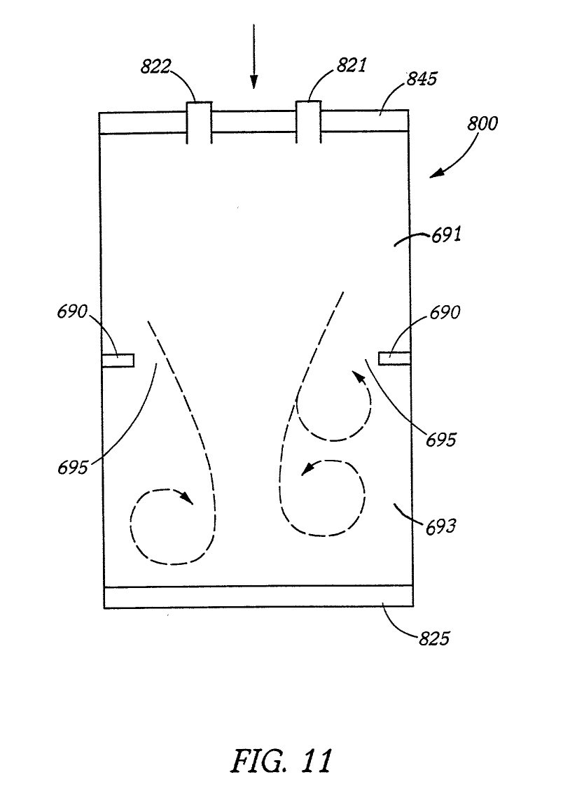

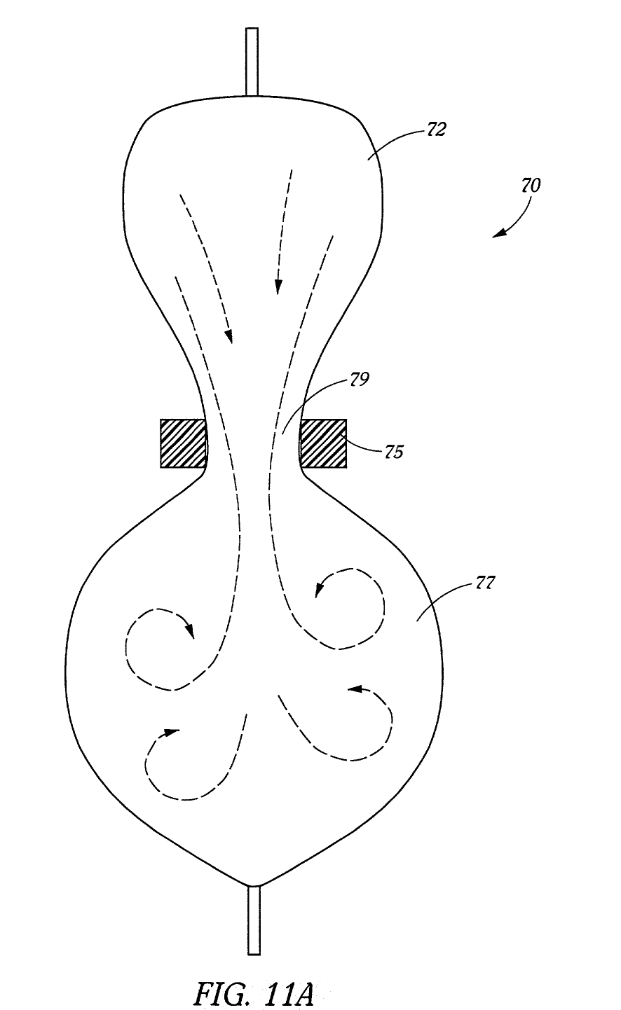

FIG. 11 shows the creation of multiple vortices within a polymeric bag similar to the type described in FIGS. 3, 8 and 9, after the opening or removal of the associated openable seal assembly. The polymeric bag 800 of FIG. 11 is shown as being constructed of a tube of polymeric film. It is understood that the bag could also be sealed or welded around all of the outer border zones as shown in FIGS. 3, 7 and 9. Only the top and bottom of the container 800 need to be sealed if the polymeric film used to make the bag is extruded in a substantially tube-like manner as described above. These outer border zones define the interior of the container. Upper seal 845 and lower seal 825 create an entirely closed container. A first side and a second side of the container are formed by the outer border zones. Two ports 821 and 822 allow fluid ingress and egress into container 800. Although only two ports are shown in FIG. 11, container 800 may have one or more ports without departing from the spirit and scope of the invention. Any well-known type of port may be used in this invention, including a port having a frangible-type mechanism. A partial seal or weld 690 is located about half way up the side of the bag, extending from the first and second sides of the bag towards the interior of the container 800. The seals or welds 690 create a narrowed or constricted portion 695 in the sides within the container 800. Upon the application of a pressure to the bag in the location of the downward pointing arrow, such as by agitation of the bag, the fluid within the container 800 is forced to move between the upper portion 691 and the lower portion 693 of the container through the narrowed portion 695 created by the seals or welds 690. This movement creates vortices within the fluid which helps to further mix the fluid. Although the vortices shown in FIG. 11 are shown as being directed towards the lower portion 693 of bag 800 respectively, the vortices could be directed to either the lower portion 693 or the upper portion 691 of the bag, depending upon the location on the bag where the force is being exerted. Preferably the solution within the bag 800 is repeatedly forced to flow between the upper portion of the container and the lower portion, to assure through mixing of solution A and solution B.

Although bag 800 above shows only partial seals 690 extending from the outer border zones of the bag, it is noted that such a bag could be divided into multiple sub-components at the approximate location of the seals by placing an openable closure assembly over the bag at such location (not shown) similar to the assemblies shown in FIGS. 3, 8 and 9. The openable closure assembly could be an external removable openable assembly, an internal openable assembly, a peelable seal, a clamp, a clip, a tongue and groove seal, or anything of the like which would removably partially divide the single bag into multiple sub-compartments and prevent communication between, or mixing of, the contents contained within each separate sub-compartment until the removable openable closure assembly is removed. It is further noted that partial seals or welds 690 such as those shown in FIG. 11 could be used in any bag including all of the bags or containers described herein.

In an alternative embodiment to the bag 800 shown in FIG. 11, the partial seals could be replaced by an external removable assembly or other structural equivalent such as a clamp, a clip, a tongue and groove seal, a vise, a clasp, a grip, or a fastener may be used to create a narrowed portion within a container. A container using such external assembly is shown in a side view in FIG. 11a, with the clamp shown in cross-section as well as the vortices created by use of the external clamp. The external clamp 75 may be used with any type of polymeric bag, including but not limited to any of the bags shown in FIGS. 3, 8, 9, 11, 12 and 14. In this embodiment, an external clamp 75 or other equivalent structure divides the container 70 into a fluidly connected upper portion 72 and a lower portion 77. Clamp 75 does not create a fluid tight seal between the upper portion 72 and the lower portion 77 of the bag. The clamp creates a narrowed portion 79 within the container 70 with which the fluid is forced through as it flows from the upper portion 72 to the lower portion 77 of container 70. Upon agitation, as the fluid within the container 70 is forced to move between the upper portion 72 and the lower portion 77 of the container through the narrowed portion 79 created by the clamp 75, vortices are created within the fluid which helps to further mix the fluid. Although the vortices shown in FIG. 11a are shown as being directed towards the lower portion 77 of bag 70 respectively, the vortices could be directed to either the lower portion 77 or the upper portion 72 of the bag, depending upon the location on the bag where the force is being exerted. Preferably the solution within bag 70 is repeatedly forced to flow between the upper and lower portion of the container, to assure through mixing of solution A and solution B.

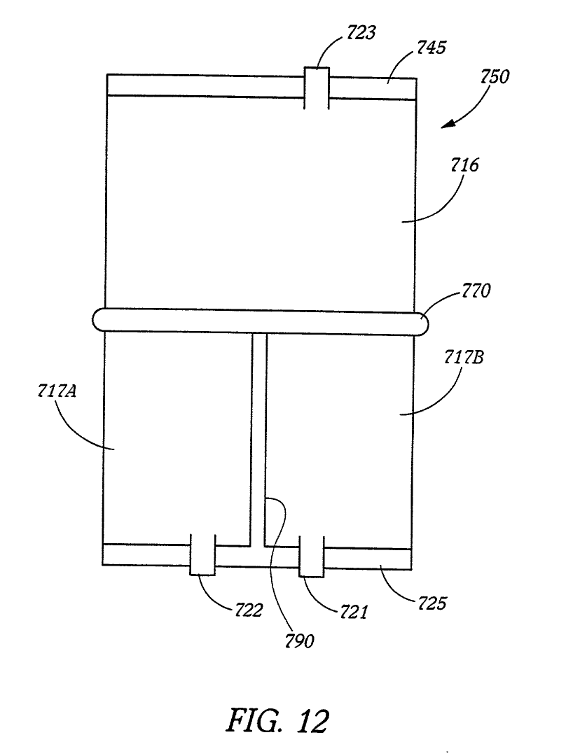

FIG. 12 shows another embodiment of a polymeric container according to the present invention wherein the openable closure assembly is a clamp. The container 750 is preferably made of polymeric type film material extruded in a tube-like shape. Because the material used to manufacture the bag is prefabricated in a tube-like shape, only two seals or welds 745 and 725 respectively, are needed to create a fluid tight, sealed interior space or main body compartment. Three ports or openings 721, 722 and 723 allow for fluid ingress and egress into and out of container 750. Port 723 allows for fluid entry or exit into upper sub-compartment 716, port 722 allows for fluid entry or exit into smaller sub-compartment 717A, and port 721 allows for fluid entry or exit into smaller sub-compartment 717B. Such ports may be located anywhere within container 750 without departing from the spirit and scope of the invention. The only requirement regulating the location of the ports in the bag 750 is that the ports be situated in a location that enables the sub-compartments to be filled via the ports. Although three ports 721, 722 and 723 respectively are shown in FIG. 11, the container 750 may have more or less ports than three without departing from the spirit and scope of the invention. Any well-known type of port may be used in this invention, including a port having a frangible-type mechanism. The main body compartment of container 750 has a partial seal or weld 790 which extends about half way up the length of the main body compartment from lower seal 725 of the container 750. The partial seal or weld 790 divides the main body portion into two partial sub-compartments 717A and 717B respectively. Although shown as extending upwards from the lower seal 725 of container 750, the partial seal or weld 790 may extend from any of the four sides of the bag 750 without departing from the spirit and scope of the invention. The main body compartment of container 750 is capable of being separated into at least three distinct sub-compartments 717A, 717B and 716 respectively, by a removable seal assembly 770. As shown in FIG. 12, in the preferred embodiment, a commercially available external clamp such as a clamp made of a glass-filled nylon material may be used. However, the removable seal assembly 770 may also be an external tongue and groove seal, an internal tongue and groove seal, a clip or anything of the like which removably divides a single bag into multiple sub-compartments and prevents communication between, or mixing of, the contents contained within each separate sub-compartment until the removable seal assembly 770 is removed. The clamp 770 is constructed of materials that will retain both shape and function at temperatures greater than or equal to 250° F., when steam sterilized with the polymeric bag 750. However, any clamp that withstands a sterilization procedure and prevents fluid leakage between sub-compartments may be used. The removable seal assembly 770 is shown extending through the center of the bag 750, dividing the bag into three sub-compartments 717A, 717B and 716. It should be noted that more than one clamp may be used to create more than three sub-compartments.

One method of using the above described embodiment in FIG. 12 may be as follows. Sub-compartment 717A may be filled with a solution containing an appropriate amount of magnesium chloride. If this embodiment is to be used for pathogen eradication, sub-compartment 717B may be filled with a solution containing an appropriate amount of riboflavin. It is also understood that either sub-compartment 717A or 717B could contain sodium chloride either alone or in combination with any component listed above. Sub-compartment 716 maybe filled with a solution containing an appropriate amount of any one or all of the following components: potassium chloride, sodium chloride, sodium acetate, monobasic sodium phosphate, dibasic sodium phosphate and/or air. These solutions are only exemplary, other components besides the above listed components that could be used in blood or blood component storage or in pathogen inactivation may be added to sub-compartments 717A, 717B or 716.

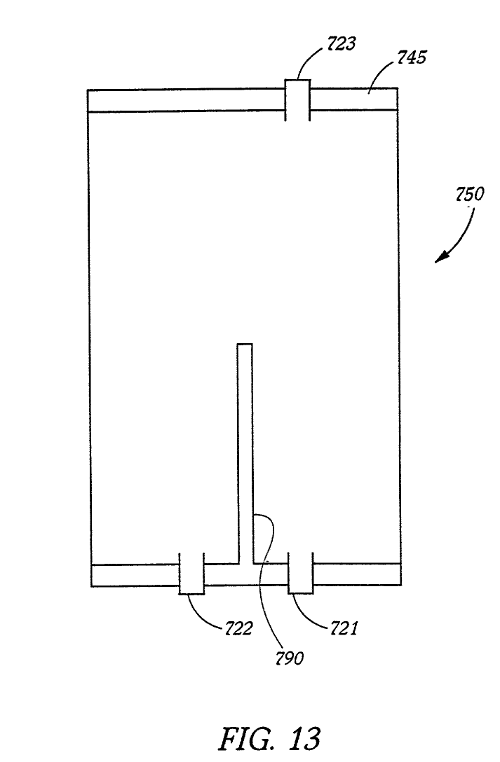

In the use of this embodiment for pathogen eradication, after sterilization of the separately stored fluids has occurred, and before the preliminary mixing of the solutions used in the pathogen inactivation process is to occur, the removable clamp 770 is removed from the container 750 (see FIG. 13.) Removal of the removable clamp 770 eliminates the seal or barrier separating the fluids within the sub-compartments, allowing the separate fluids to combine into a single combined solution. After combination of the fluids, the blood or blood component to be pathogen inactivated may be added to container 750 via ports 723, 722 or 721. Alternatively, the combined fluid may be added to a second bag (not shown) containing the blood or blood component to be pathogen inactivated. The blood or blood component or the combined fluid to which the blood or blood component is added and the pathogen inactivation solution are mixed together, and the mixture may be exposed to a light source. As noted above, this arrangement can also be used with partial seals similar to element 690 shown in FIG. 11 to enhance the mixing of the fluid. It should be noted that if a second bag is used, the bag could be in a configuration such as that described in FIG. 11.

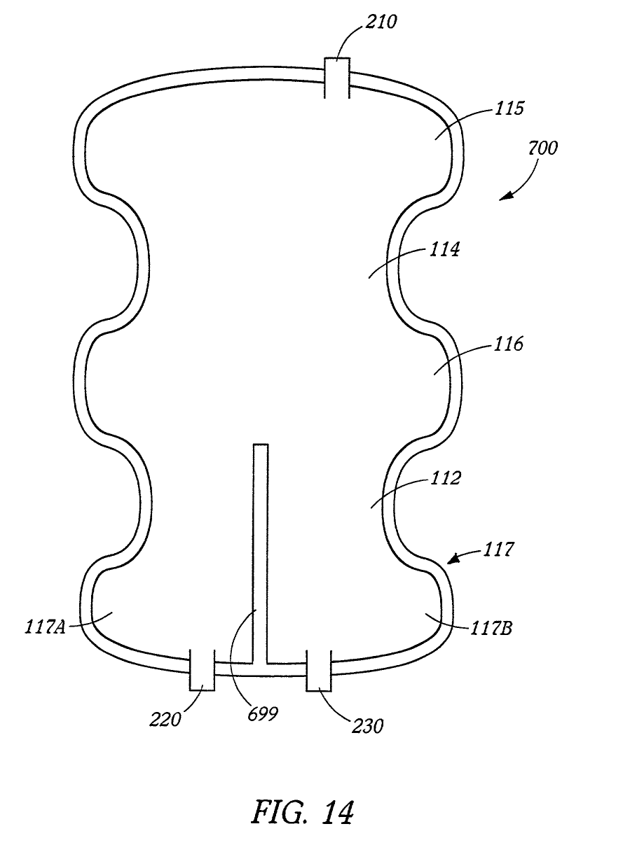

FIG. 14 shows a top view of another alternative embodiment of a further polymeric container 700 according to the present invention. This container 700 is preferentially made of a polymeric type film such as polyolefin. The container is sealed or welded around its outer border zones during manufacture. The seals or welds create a fluid tight, sealed interior space. The container is configured in a double figure eight or double hourglass shape as shown. The container 700 has an upper expanded interior or sub-compartment portion 115, a middle expanded interior or sub-compartment portion 116, and a lower expanded interior portion or sub-compartment 117 defined by its sides. The upper expanded sub-compartment 115 and the middle expanded sub-compartment 116 are connected to each other in a fluidly communicative relationship by narrowed portion 114 defined by its sides. The middle expanded sub-compartment 116 and the lower expanded sub-compartment 117 are also connected to each other in a fluidly communicative relationship by narrowed portion 112 defined by the side of the container. The lower expanded sub-compartment 117 is divided into two smaller sub-compartments 117A and 117B by an interior seal or weld 699. Both the upper expanded sub-compartment 115 and the middle expanded sub-compartment 116 may also be further sub divided into multiple sub-compartments (not shown) without departing from the spirit and scope of the invention. Three ports or openings 210, 220 and 230 allow for fluid ingress and egress into and out of container 700. Port 210 allows for fluid entry or exit into upper expanded sub-compartment 115, port 220 allows for fluid entry or exit into smaller sub-compartment 117A, and port 230 allows for fluid entry or exit into smaller sub-compartment 117B. Such ports may be located anywhere within container 700 without departing from the spirit and scope of the invention. The only requirement regulating the location of the ports in the bag 700 is that the ports be situated in a location that enables the sub-compartments to be filled via the ports. Although only three ports 210, 220 and 230 respectively are shown in FIG. 14, the container 700 may have more or less than three ports. Any well-known type of port may be used in this invention, including a port having a frangible-type mechanism.

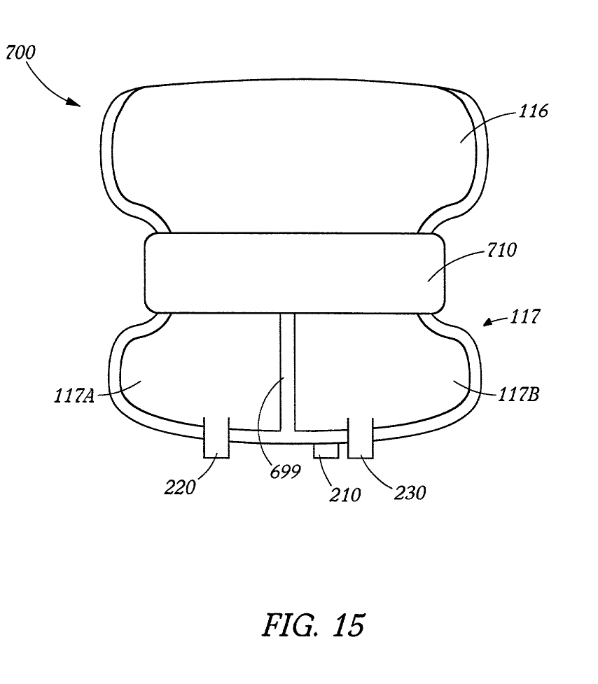

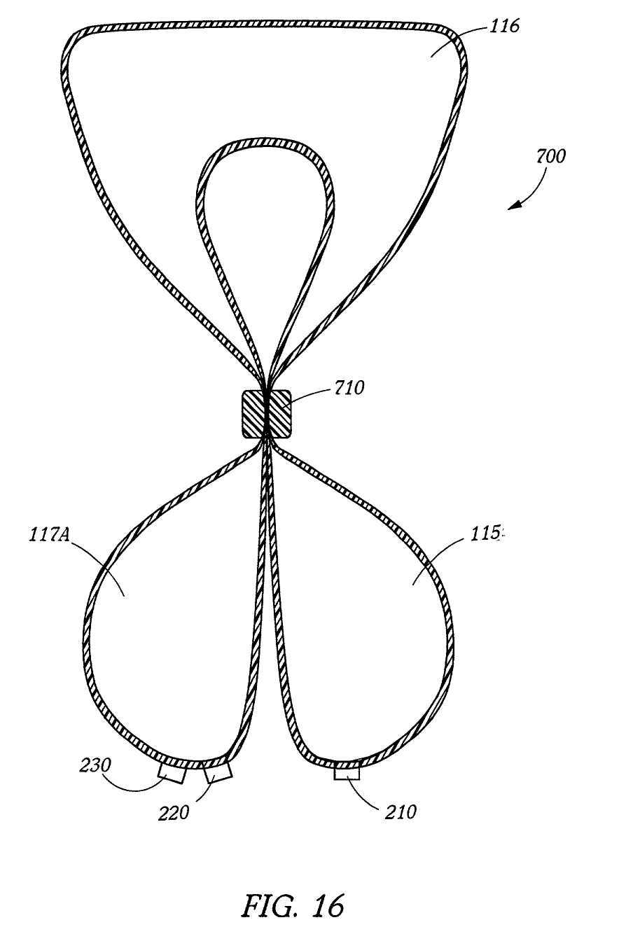

As shown in FIGS. 15 and 16, a commercially available clamp 710 such as that described in FIG. 12 above, may be placed on the bag 700 in such a fashion as to divide bag 700 into four separately contained fluid-tight or fluidly separated sub-compartments. The separately contained sub-compartments correspond to sub-compartments 117B, 116, 115 and 117A respectively. Sub-compartment 115 (seen in FIG. 14) is not seen in the particular depiction of this embodiment shown in FIG. 15. Sub-compartment 115 is located behind sub-compartment 117 due to the location of the clamp 710. Sub-compartment 116 between sub-compartment 115 and sub-compartments 117A and 117B (117B seen in FIG. 14) is not seen in the particular depiction of this embodiment shown in FIG. 16) may be used as a visual indicator to determine if fluids contained within separate sub-compartments 117A, 117B or 115 have leaked into other compartments during the storage or sterilization process.

The bag in FIGS. 14, 15 and 16 is capable of storing components necessary for blood storage or for photoinactivation of pathogens or other contaminants within the blood or blood components, as will be described below. Middle sub-compartment 116 of container 700 may be filled with a fluid such as air, and also may serve as a quality control indicator of the tightness of the seal made by clamp 710. Middle component 116 may also contain fluid such as oxygen or nitric oxide. Upper sub-compartment 115 for example, may be filled with a fluid containing a photosensitizer. Smaller sub-compartment 117A may be filled with another type of fluid such as a buffer. Smaller sub-compartment 117B may contain a fluid such as a magnesium chloride solution or a potassium chloride solution or both, but is not seen in the cross-sectional depiction of FIG. 16 as noted above since smaller sub-compartment 117B is located on the other side of smaller sub-compartment 117A. It should be noted however that the sub-compartments may contain a solid or a powdered material without departing from the spirit and scope of the invention.

In one use of the embodiment described in FIGS. 14, 15 and 16, container 700 is initially filled with a fluid such as air via port 210, 220 or 230. When initially added to the container, the air fills the entire container. In order to create the sub-compartments within the bag using a clamp, the air must be expressed from the other portions 115, 117 of the container into the middle portion 116. The bag 700 is then folded in half across the middle expanded portion 116 and clamped with clamp 710 across the narrowed portions 114 and 112 of the bag 700, to divide the bag into four fluid tight or fluidly separated sub-compartments, 115, 116, 117A and 117B respectively (shown in FIGS. 15 and FIG. 16). Upper expanded portion 115 may be filled with a photoinactivation fluid such as riboflavin which is flowed into sub-compartment 115 via port 210. Smaller sub-compartment 117A may be filled with a fluid such as a buffer solution which is flowed into smaller sub-compartment 117A via port 220. Smaller sub-component 117B may be filled with a fluid such as a magnesium chloride solution which is flowed into smaller sub-component 117B via port 230. The middle expanded portion 116, which is filled with air serves as a visual indicator of the presence of fluid leaks between the multiple fluid filled compartments. The air filled compartment may also aid in oxidation reactions that may take place within the container 700, after the removal of clamp 710.

In the use of this embodiment, after sterilization of the separately stored fluids has occurred, and before the preliminary mixing of the solutions used in the pathogen inactivation process is to occur, the removable clamp 710 is removed from the container 700. Removal of the removable clamp eliminates the seal or barrier separating the fluids within the sub-compartments, allowing the combination of the separate fluids into a single combined fluid containing photosensitizer, buffer, magnesium chloride solution and air (shown in FIG. 14). These solutions are only exemplary, other components besides the above-listed components that could be used in blood or blood component storage, or in pathogen inactivation, may be added to the sub-compartments. After combination of the fluids, the blood or blood component to be pathogen inactivated may be added to container 700 via port 210, 220 or 230. Alternatively, the combined fluid may be added to the blood or blood component to be pathogen inactivated. The blood or blood component to be pathogen inactivated and the pathogen inactivation solution are mixed together, and the mixture may be exposed to a light source. The mixture may optionally be agitated to disperse the photosensitizer evenly throughout the container 700.

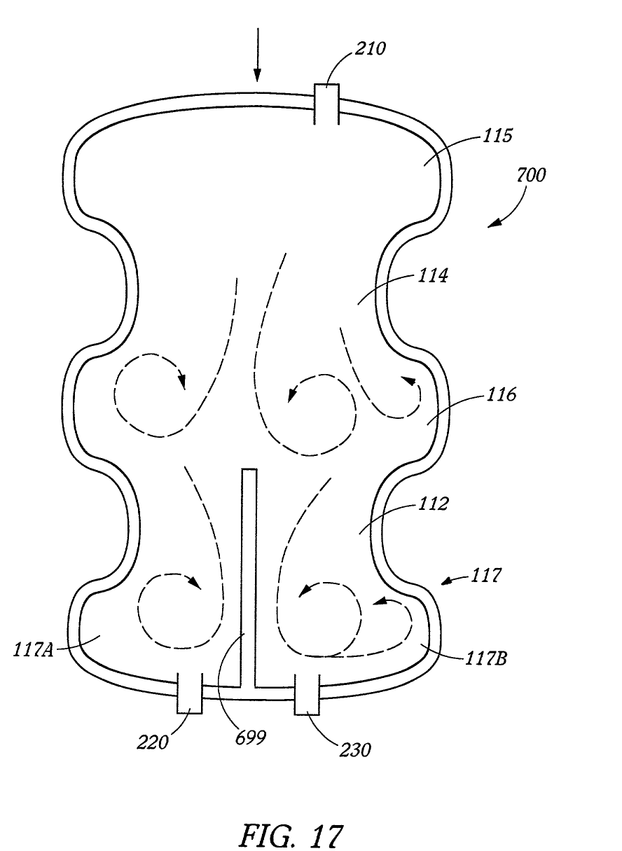

As shown in FIG. 17, the double figure eight or double hourglass configuration may aid in mixing the solution within the bag 700. The fluid in the bag may be mixed in a substantially vertical manner or a substantially horizontal manner and/or both. A force imparted to the bag at a particular location (depicted by the downward arrow) creates the initial movement of fluid within the bag. As the fluid is forced to move through the narrowed portions 114 and 112 of the double hourglass shape, vortices are created within the fluid which helps to further mix the fluid. The seal or weld 699 which separates the lower expanded portion 117 into two smaller sub-compartments 117A and 117B may also assist in further creating vortices within the fluid. A clamp (not shown) such as that described in Fig. 11a may also be used to create narrowed portions within the bag. The narrowed portion or portions create vortices within the fluid as the fluid is forced to flow through the narrowed portions.

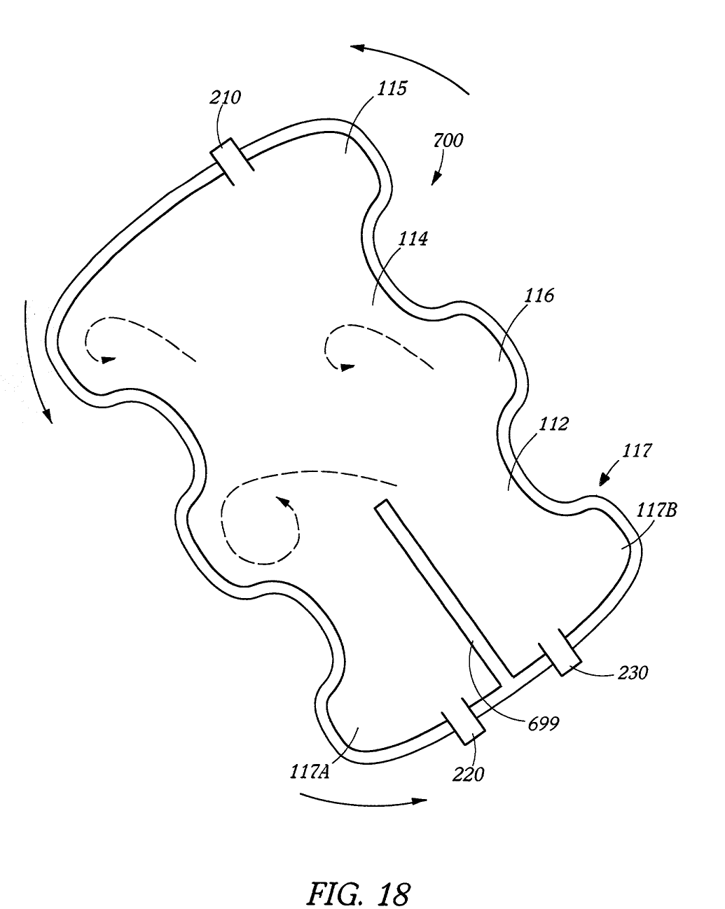

FIG. 18 shows the bag 700 in the double figure eight or double hourglass configuration being mixed by rotation in either a substantially vertical and/or a substantially horizontal manner. The bag may be rotated about its centerpoint between about 180° and about 360° in a continuous fashion. A force (from the rotation itself and/or due to the force of gravity when in a vertically disposed embodiment) may be imparted to the bag by virtue of the rotation of the bag to create the initial movement of fluid within the bag. As the fluid is forced to move around the double figure eight or double hourglass shape, vortices are created within the fluid which helps to further mix the fluid. The bag may be rotated in a continuous manner, or may be agitated in varying degrees from between about 0° to about 360° without departing from the spirit and scope of the invention. The bag may also be rotated either singly or in a repetitive manner.

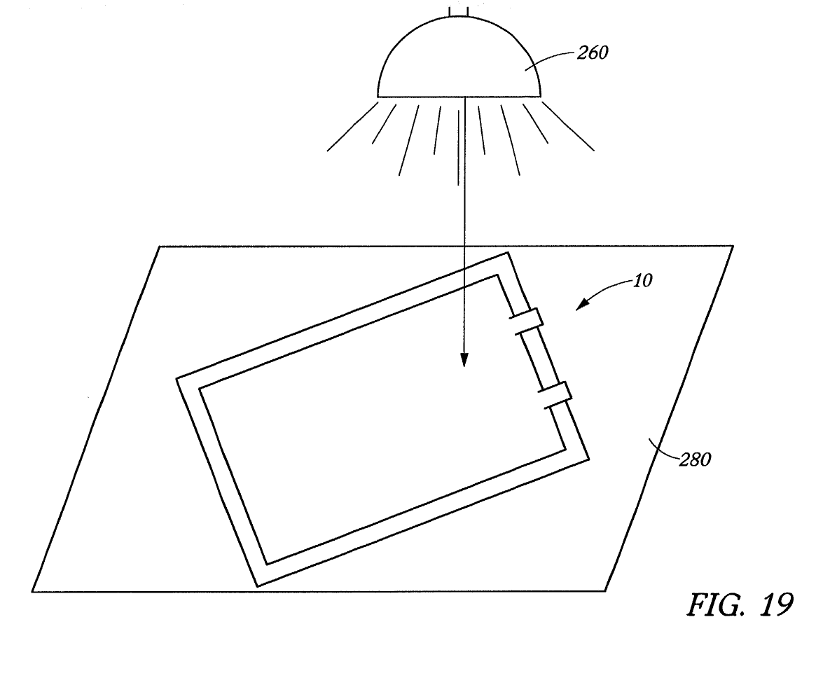

The blood component to be pathogen inactivated which may be contained within any of the aforementioned bags collected using the apparatus shown in FIGS. 1 and 2, may be added to the single combined solutions as described with reference to FIGS. 3, 8, 9, 11, 12 and 14. In the preferred embodiment, it is envisioned that platelets will be the blood component pathogen inactivated using this method, however, any blood component or whole blood may also be used with this invention. Alternatively, the combined solution can be transferred out of any of the aforementioned bags of the invention into another container or bag or into bag 80, 90 or 950 (shown in FIG. 2) before pathogen inactivation. At the time of solution transfer into bag 80, 90 or 950 the bag may or may not contain blood or blood components to be pathogen inactivated. After addition of the blood component to be pathogen inactivated, the bag containing the photosensitizing solution is irradiated, as shown in FIG. 19 (with bag 10 of FIG. 3 as an example). Shaker table 280 is optionally agitated to disperse the solution containing photosensitizer and the blood component to be irradiated evenly throughout the container 10, while the radiation source 260 irradiates the photosensitizer to inactivate pathogens contained within the blood component. It is also understood that other known methods of irradiation may be used and that shaker table agitation may be optional.

The examples of the above-described polymeric bags having openable seal assemblies are for illustrative purposes only. Because of variations which will become apparent to those skilled in the art, the present invention is not meant to be limited to the particular embodiments described above. Any such variations and other modifications or alterations are included within the scope and intent of the invention.

1. A single container capable of being divided into multiple sub-compartments for use in pathogen inactivation of blood or blood components comprising at least one outer border zone which defines the interior of the container;
a first sub-compartment adapted for containing a first fluid comprising a photosensitizer;
a second sub-compartment adapted for containing a second fluid;
an openable seal assembly which openably fluidly separates the first sub-compartment from the second sub-compartment whereby opening of the openable seal assembly allows mixing of the first fluid with the second fluid.

2. The container of claim 1 wherein the first fluid further comprises a nutrient.

3. The container of claim 1 wherein the photosensitizer comprises an isoalloxazine.

4. The container of claim 1 wherein the photosensitizer comprises riboflavin.

5. The container of claim 2 wherein the nutrient comprises glucose.

6. The container of claim 1 wherein the second fluid comprises a buffer.

7. The container of claim 6 wherein the second fluid further comprises an anticoagulant.

8. The container of claim 1 wherein the openable seal assembly further comprises
a channel; and
a removable spline wherein the removable spline is adapted for insertion and removal into the channel.

9. The container of claim 8 wherein a portion of the container is inserted into the channel and held within the channel by the removable spline when the removable spline is inserted into the channel to form the fluidly separate first and second sub-compartments.

10. The container of claim 8 wherein the removable spline has ridges along its outermost surface to aid in fluidly separating the first and second sub-compartments when the removable spline is inserted into the channel.

11. The container of claim 8 wherein the removable spline has a hole disposed longitudinally therethrough to provide compressibility to the spline to aid in fluidly separating the first and second sub-compartments when the removable spline is inserted into the channel.

12. The container of claim 1 wherein the openable seal assembly further comprises
a tongue; and
a groove wherein the tongue is adapted for insertion and removal into the groove.

13. The container of claim 12 wherein a portion of the container may be inserted into the groove and held within the groove by the tongue when the tongue is inserted into the groove to form the fluidly separate first and second sub-compartments.

14. The container of claim 12 wherein the tongue is made of a substantially rigid material.

15. The container of claim 14 wherein the groove is made of a substantially flexible material which is adapted to accommodate the container and the rigid tongue to form fluidly separated sub-compartments when the rigid tongue is inserted into the groove.

16. The container of claim 1 wherein the openable seal assembly comprises
an internal tongue in the interior of the container; and
an internal groove in the interior of the container wherein the internal tongue is adapted for insertion and removal into the internal groove.

17. The container of claim 16 wherein the internal tongue and internal groove extends the length of the container through the interior of the container to fluidly separate the first sub-compartment from the second sub-compartment when the internal tongue is inserted into the internal groove.

18. The container of claim 16 further comprising
a first interior surface;
a second interior surface;
a first exterior surface opposite the first interior surface; and
a second exterior surface opposite the second interior surface wherein the internal tongue is affixed to the first internal surface and the internal groove is affixed to the second interior surface of the container opposite the internal tongue.

19. The container of claim 18 further comprising
a first tab adapted to be affixed to the first exterior surface of the container; and
a second tab adapted to be affixed to the second exterior surface of the container wherein the tabs can be manually grasped to separate the internal tongue from the internal groove.

20. The container of claim 1 farther comprising at least one seal or weld extending from at least one of the outer border zones toward the interior of the container to form a third sub-compartment within the interior of the container and wherein the openable seal assembly fluidly separates the first sub-compartment, the second sub-compartment and the third sub-compartment.

21. The container of claim 1 capable of being divided into multiple sub-compartments for pathogen inactivating blood or blood components further comprising;
a third sub-compartment fluidly interconnected with the second sub-compartment wherein the third sub-compartment is adapted for containing a third fluid; and
a fourth sub-compartment fluidly interconnected with the second sub-compartment and adjacent to the third sub-compartment wherein the fourth sub-compartment is adapted for containing a fourth fluid.

22. The container of claim 21 further comprising at least one seal adapted to extend from the at least one outer border zone into the interior of the container a distance of about half way up the length of the container to separate the third sub-compartment from the fourth sub-compartment.

23. The container of claim 21 wherein the second fluid comprises air.

24. The container of claim 21 wherein the third fluid comprises an additive.

25. The container of claim 21 wherein the fourth fluid comprises an additive.

26. The container of claim 25 wherein the additive comprises magnesium chloride.

27. The container of claim 21 wherein the container is adapted to be in a double hourglass shape.

28. The container of claim 21 further comprising
a first narrowed portion;
a second narrowed portion;
a first expanded portion adjacent to and fluidly connected with the first narrowed portion;
a second expanded portion adjacent to and fluidly connected with the first narrowed portion and adjacent to and fluidly connected with the second narrowed portion; and
a third expanded portion adjacent to and fluidly connected with the second narrowed portion.

29. The container of claim 28 wherein the narrowed portions are adapted to engage with the openable seal assembly.

30. The container of claim 29 wherein the openable seal assembly comprises a clamp for engaging the container to fluidly separate the expanded portions to form the first, second, third and fourth sub-compartments.

31. A single container for use in mixing a fluid comprising
at least one outer border zone which defines the interior of the container;
a first side formed by the outer border zone;
a second side formed by the outer border zone wherein the first and second sides define a first narrowed portion in the interior of the container.

32. The container of claim 31 wherein the first and second sides further define a first expanded portion adjacent to and fluidly connected with the first narrowed portion.

33. The container of claim 31 wherein the first narrowed portion helps create vortices in the fluid when the fluid in mixed.

34. The container of claim 31 wherein the first narrowed portion comprises first and second seals extending from the outer border zone.

35. The container of claim 34 wherein the first seal extends from the outer border zone opposite the second seal.

36. The container of claim 34 wherein the first and second seals help create vortices in the fluid when the container is mixed.

37. The container of claim 31 wherein the first and second sides further define
a second narrowed portion adjacent to and fluidly connected with the first expanded portion;
a second expanded portion adjacent to and fluidly connected with the first narrowed portion and adjacent to and fluidly connected with the second narrowed portion; and
a third expanded portion adjacent to and fluidly connected with the second narrowed portion.

38. The container of claim 37 wherein the first and second narrowed portions help create vortices in the fluid when the fluid in mixed.

39. The container of claim 37 wherein the first and second narrowed portions are adapted to engage with at least one openable seal assembly to form a first sub-compartment and a second sub-compartment.

40. The container of claim 39 wherein the openable seal assembly comprises a clamp for engaging the container to fluidly separate the sub-compartments.

41. A method for inactivating a fluid which may contain pathogens comprising the steps of
providing a first container;
flowing a first fluid through a first port into a first sub-compartment of the first container;
flowing a second fluid through a second port into a second sub-compartment of the first container;
opening an openable seal assembly between the first sub-compartment and the second sub-compartment to allow fluid to flow between the sub-compartments;
combining the first fluid with the second fluid to form a first fluid mixture; and
combining a blood component with the first fluid mixture to form a second fluid mixture.

42. The method of claim 41 further comprising photoinactivating the second fluid mixture.

43. The method of claim 41 wherein one of the first or second fluids contains riboflavin.

44. The method of claim 41 wherein the step of combining comprises
adding the first fluid mixture to a second container containing blood or blood component to be photoinactivated; and
mixing the blood component and first fluid mixture to form the second fluid mixture.

45. The method of claim 41 wherein the step of combining comprises adding the blood component to the first fluid mixture in the first container.

46. The method of claim 44 further comprising photoinactivating the second fluid mixture.

47. The method of claim 44 wherein one of the first or second fluids contains riboflavin.

48. A single container capable of being divided into multiple sub-compartments for use in pathogen inactivation of blood or blood components comprising
at least one outer border zone which defines the interior of the container;
a first sub-compartment adapted for containing a photosensitizer;
a second sub-compartment adapted for containing a component wherein at least one of the photosensitizer or component is in solid form;
an openable seal assembly which openably separates the first sub-compartment from the second sub-compartment whereby opening of the openable seal assembly allows mixing of the photosensitizer and component.

49. The container of claim 48 wherein both the photosensitizer and component are in solid form.

50. The container of claim 48 wherein the photosensitizer comprises an isoalloxazine.

51. The container of claim 48 wherein the component comprises a buffer.
